(19) United States
(12) Patent Application Publication
(10) Pub. No.: US 2023/0309830 A1
(43) Pub. Date: Oct. 5, 2023

(54) SYSTEM AND METHOD FOR PROVIDING A REMEDIAL PROCEDURE

(71) Applicant: Akash Madnani, Nagpur (IN)

(72) Inventor: Akash Madnani, Nagpur (IN)

(21) Appl. No.: 18/325,102

(22) Filed: May 29, 2023

Related U.S. Application Data
(63) Continuation of application No. 16/609,535, filed on Oct. 30, 2019, now abandoned.

(30) Foreign Application Priority Data
May 8, 2017 (IN) 201721016237

(51) Int. Cl. A61B 5/00; A61B 5/0205; A61B 5/11

(52) U.S. Cl. CPC A61B 5/0022; A61B 5/0205; A61B 5/1118; A61B 2562/0219

(57) ABSTRACT

A system, a method, and a computer program product may be provided for providing a remedial procedure. The system comprises a wearable device having sensors to measure health parameters of a user, and processors connected to the wearable device. The processors may receive the health parameters from the sensors, determine a physical wellness value for the user based on the health parameters and predefined physical wellness thresholds, and determine a mental wellness value for the user based on the health parameters and predefined mental wellness thresholds. The processors may identify a risk factor associated with a health of the user based on the physical wellness value and the mental wellness value. The processors may provide a remedial procedure in real-time designed to at least mitigate the identified risk factor.

### TECHNOLOGICAL FIELD

The present disclosure generally relates to monitoring human performance, and more particularly, it relates to systems and methods for monitoring human performance and providing remedial procedures to improve health.

### BACKGROUND

In recent years, there has been a rapid increase in the number of people suffering from stress and heart-related conditions. Typically, health parameters of a human are used to identify the onset of health risks such as stress and heart-related conditions.

Diagnosis of health risks is often limited to clinical or laboratory settings, which may be inconvenient for some people. As a result, individuals may fail to identify the early signs of a disease or health risk, such as those caused by stress or heart-related issues.

### SUMMARY

In one aspect, a system for providing a remedial procedure is provided. The system comprises a wearable device configured to operate with one or more sensors to measure a set of health parameters of a user. The health parameters comprise at least one of: breathing data, state of mind data, activity data, posture data, sleep pattern data, and ambience data. The system further comprises one or more processors operably connected to the wearable device. The one or more processors are configured to receive the set of health parameters of the user from the one or more sensors, determine a physical wellness value for the user, and determine a mental wellness value for the user. The physical wellness value is determined based on the set of health parameters and a set of predefined physical wellness thresholds. The physical wellness value is associated with at least one of: a breathing pattern, or a heart rate, of the user. The mental wellness value is determined based on the set of health parameters and a set of mental physical wellness thresholds. The mental wellness value is associated with at least a stress condition of the user. The one or more processors are configured to identify a risk factor associated with a health of the user based on the physical wellness value and the mental wellness value. The one or more processors are configured to provide the remedial procedure in real-time designed to at least mitigate the identified risk factor.

In accordance with an example embodiment, the remedial procedure is a time-based remedial process that is triggered based on the identification of the risk factor.

In accordance with an example embodiment, the remedial procedure is determined to regulate at least one of: a heart rate variability (HRV), or a blood pressure, of the user.

In accordance with an example, the remedial procedure is determined based on the set of health parameters of the user. For example, based on the set of health parameters, specifically, the breathing data, the state of mind data, the activity data, the posture data, the sleep pattern data, and the ambience data, the HRV and the blood pressure of the user may be identified. Further, the HRV of the user may be increased and the blood pressure of the user may be reduced to regulate wellness value of the user.

Some embodiments of the present disclosure are based on recognition that the HRV of the user is an important health parameter to gauge the physical health and the mental health of the user.

In accordance with an example embodiment, the determined remedial procedure is associated with a resonant breathing activity. For example, the resonant breathing activity comprises a low deep breathing exercise at 6 to 8 breaths per minute (bpm). In an example, by causing the user to perform the resonant breathing activity, the HRV and/or the blood pressure of the user may be regulated to a healthy state.

In accordance with an example embodiment, the one or more processors are further configured to cause the user to perform the resonant breathing activity in order to address the risk factor relating to at least one of: heart rate, heart rate variability (HRV), free radicals, or stress regulating system. For example, the stress regulating system is associated with autonomic nervous system (ANS).

In accordance with an example embodiment, one or more processors are further configured to output a set of instructions to display. The set of instructions relate to the resonant breathing activity.

In accordance with an example embodiment, one or more processors are further configured to retrieve health condition management data relating to one or more users. The one or more users have the risk factor identified for the user. The one or more processors are further configured to determine the remedial procedure relating to the risk factor based on the health condition management data, the physical wellness value, the mental wellness value, a set of historical mental wellness values of the user, and a set of physical wellness values of the user. The remedial procedure comprises personalized wellness data for the user. The one or more processors are further configured to output a notification to display based on the determined remedial procedure. The notification indicates the remedial procedure for improving at least one of: the physical wellness value, or the mental wellness value, of the u ser.

In accordance with an example embodiment, the one or more processors are further configured to retrieve the set of historical physical wellness values associated with the user, and determine the physical wellness value for the user based on the set of health parameters and the set of historical physical wellness values.

In accordance with an example embodiment, the one or more processors are further configured to retrieve the set of historical mental wellness values associated with the user, and determine the mental wellness value for the user based on the set of health parameters and the set of historical mental wellness values.

In accordance with an example embodiment, the one or more processors are further configured to determine the mental wellness value for the user based on the breathing data and the state of mind data.

In accordance with an example embodiment, the one or more processors are further configured to periodically determine the physical wellness value and the mental wellness value of the user, and monitor at least one of: physical wellness, or mental wellness, of the user based on at least one of: the physical wellness value, or the mental wellness value. The one or more processors are further configured to output an indicator for the user in real-time. The indicator relates to at least one of: breathing, or stress, based on at least one of the: physical wellness, or mental wellness.

In accordance with an example embodiment, the one or more sensors comprise at least one of: a light sensor, a noise sensor, a temperature sensor, an accelerometer, a gyroscope, a magnetometer, or a strain gauge.

In accordance with an example embodiment, the wearable device is worn on a torso of the user.

In another aspect, a method for providing a remedial procedure is provided. The method comprises receiving a set of health parameters of the user from one or more sensors. The health parameters comprise at least one of: breathing data, state of mind data, activity data, posture data, sleep pattern data, and ambience data. The method comprises determining a physical wellness value for the user based on the set of health parameters and a set of predefined physical wellness thresholds and determining a mental wellness value for the user based on the set of health parameters and a set of mental physical wellness thresholds. The physical wellness value is associated with at least one of: a breathing pattern, or a heart rate, of the user. The mental wellness value is associated with at least a stress condition of the user. The method comprises identifying a risk factor associated with the health of the user based on the physical wellness value and the mental wellness value. The method comprises providing a remedial procedure in real-time designed to at least mitigate the identified risk factor.

In yet another aspect, a computer programmable product for providing a remedial procedure is provided. The computer programmable product comprises a non-transitory computer readable medium having stored thereon computer executable instructions, which when executed by one or more processors, cause the one or more processors to carry out operations. The operations comprise receiving a set of health parameters of the user from one or more sensors. The health parameters comprise at least one of: breathing data, state of mind data, activity data, posture data, sleep pattern data, and ambience data. The operations comprise determining a physical wellness value for the user based on the set of health parameters and a set of predefined physical wellness thresholds and determining a mental wellness value for the user based on the set of health parameters and a set of mental physical wellness thresholds. The physical wellness value is associated with at least one of: a breathing pattern, or a heart rate, of the user. The mental wellness value is associated with at least a stress condition of the user. The operations comprise identifying a risk factor associated with the health of the user based on the physical wellness value and the mental wellness value. The operations comprise providing a remedial procedure in real-time designed to at least mitigate the identified risk factor.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the disclosure in general terms, reference will now be made to the accompanying drawings, which are not necessarily drawn to scale, and wherein:

FIG. 1 shows a block diagram of a system for providing a remedial procedure, in accordance with an embodiment;

FIG. 2 illustrates a back-perspective view of a wearable device, in accordance with an embodiment;

FIG. 3 illustrates a front-perspective view of the wearable device, in accordance with an embodiment;

FIG. 4 illustrates a provisioning of the wearable device associated with a user, in accordance with an embodiment;

FIG. 5 illustrates a sectional view of the wearable device, in accordance with an embodiment;

FIG. 6 illustrates an arrangement of the wearable device in a vibrating mode, in accordance with an embodiment;

FIG. 7A, FIG. 7B, and FIG. 7C illustrate various positions for arranging the wearable device associated a user, in accordance with various embodiments;

FIG. 8A, FIG. 8B, and FIG. 8C illustrate various postures measured by the wearable device for calibration, in accordance with various embodiments;

FIG. 9 illustrates a flowchart of transmitting a set of health parameters to processors for processing, in accordance with an embodiment;

FIG. 10 illustrates a working flowchart for processing breathing data of the user, in accordance with an embodiment;

FIG. 11 illustrates a waveform of a breathing pattern of the user, in accordance with an embodiment;

FIG. 12 illustrates a working flowchart of processing posture data and activity data of the user, in accordance with an embodiment;

FIG. 13 shows a working flowchart for identifying sleep pattern data and ambience data around the user in the night, in accordance with an embodiment;

FIG. 14 illustrates a working flowchart for identifying a risk factor for the user, in accordance with an embodiment;

FIG. 15 illustrates a flowchart of generating a notification for providing a remedial procedure to a user, in in accordance with an embodiment;

FIG. 16 shows a method for monitoring human performance, in accordance with an embodiment;

FIG. 17 shows a method for providing a remedial procedure to the user, in accordance with an embodiment; and

FIG. 18 illustrates a schematic diagram for performing operations of a system for providing a remedial procedure, in accordance with an embodiment.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, devices and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present disclosure. Thus, use of any such terms should not be taken to limit the spirit and scope of embodiments of the present disclosure.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

Embodiments of the present disclosure provide a system, a method, and a computer programmable product for providing a remedial procedure for mitigating a health risk factor of a user. As may be understood, an increasing number of people are suffering from physical and mental health issues putting them at risk for various acute and chronic health-related risks. In order to ensure the well-being of people, it becomes crucial to inform them about their health parameters. This may be achieved by wearables such as smart watches that monitor a person's health parameters, such as heart rate, number of steps, sleep quality, etc.

However, simply having an awareness of one's health parameters may not be enough to adequately improve their overall health. For example, understanding one's heart rate and stress levels may provide valuable insight, it does not necessarily translate into improved cardiac or mental health. Thus, a measurable call-to-action is necessary if any health parameters detected are sub-optimal. Unfortunately, traditional monitoring devices, such as smart watches and fitness trackers, do not have the capability of providing users with measurable actionable steps to improve their health status. Without this support, users may be unable to make the necessary lifestyle changes in order to achieve improved health outcomes.

To this end, an object of the present disclosure is to provide a system and a method for monitoring health parameters of a user and for providing a remedial procedure for improving the user's health. The health parameters of the user are monitored to keep real-time track of mental wellness and physical wellness of the user. Based on identifying low or poor mental wellness and physical wellness of the user, a risk factor is identified for the user in a particular period of time.

It is an object of the present disclosure is to provide a system and a method for providing a remedial procedure to the user to help the user to regulate their physical and mental wellness.

Another object of the present disclosure is to provide a system and a method for sending an alert to the user when their evaluated health parameters are not in correlation with certain thresholds associated with the health parameters. The system and method disclosed herein may assist and monitor the user in real time for improving their health.

The present disclosure provides a system and a method for monitoring health parameters of a user and providing a remedial procedure. The system may include a wearable device that keeps real-time track of health parameters of the user. Further, the system may include processors that are configured to identify any deviation or anomaly associated with the health parameters of the user to identify any physical or mental problem, i.e., a risk factor for the user in a particular period of time. Further, the processors may identify a remedial procedure for improving the health of the user based on the risk factor. The remedial procedure may be a time-based remedial process that is triggered based on the identification of the risk factor. The processors may be configured to send an alert to the user when the risk factor is identified and assist them in performing the remedial procedure by providing set of instructions.

The disclosed embodiments are merely exemplary of the invention, which may be embodied in various forms.

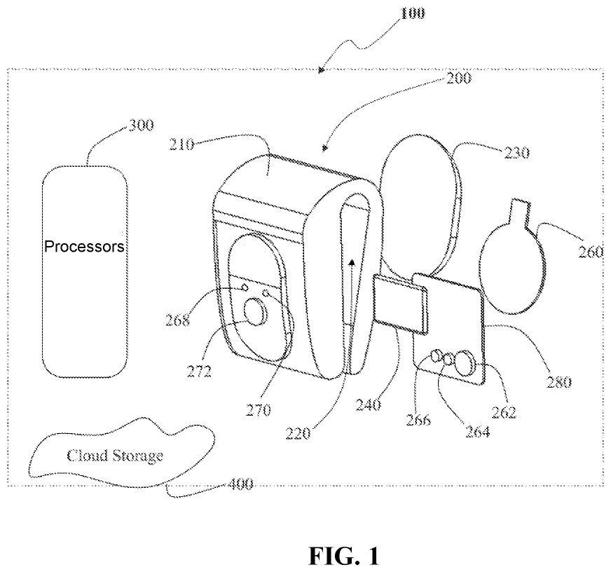

Referring now to FIG. 1, a block diagram of a system 100 for providing remedial procedure is shown, in accordance with an example embodiment. The system 100 includes a wearable device 200, one or more processors 300 (referred to as processors 300, hereinafter) and a cloud storage 400. In an example, the processors 300 may be embodied within the wearable device 200. In another example, the processors 300 may be remotely located and coupled to the wearable device 200, via a communication interface. For example, the communication interface may be wired or wireless.

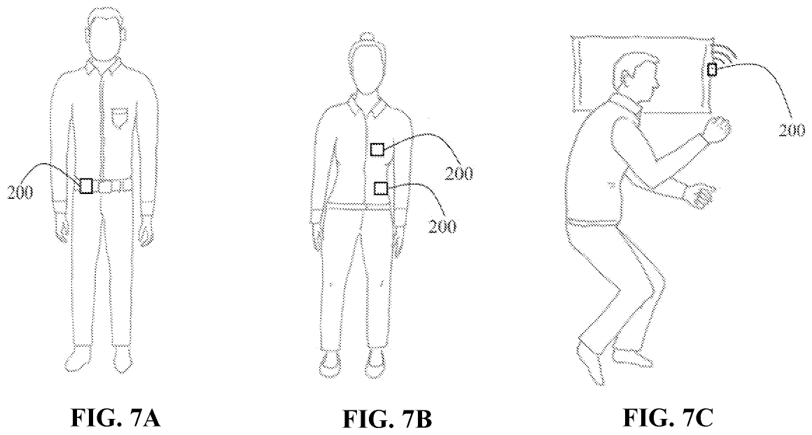

The wearable device 200 may be worn anywhere on the torso of a user. For example, the wearable device 200 may be secured over inner wears of the user. Specifically, a male user may wear the wearable device 200 near their waist as shown in FIG. 7A and a female user may wear the wearable device 200 on their clothing at their waist, or on their torso near their breast or on a bra as shown in FIG. 7B. In certain cases, the wearable device 200 may be positioned on a pillow on which the user is resting their head while sleeping as shown in FIG. 7C. It may be obvious to a person skilled in the art to wear or position the wearable device 200 on any other portions of a torso of the user.

In operation, the wearable device 200 is configured to operate with one or more sensors to measure a set of health parameters of a user. The health parameters may include, but are not limited to, breathing data, state of mind data, activity data, posture data, sleep pattern data, and ambience data.

Further, the processors 300 are configured to receive the set of health parameters of the user from one or more sensors. The processors 300 are configured to determine a physical wellness value for the user. In an example, the physical wellness value may be determined based on the set of health parameters and a set of predefined physical wellness thresholds. Specifically, the physical wellness value may be determined based on the breathing data of the user and the predefined physical wellness thresholds indicating a healthy or normal number of breaths per minute. The physical wellness value is associated with a breathing pattern, or the heart rate of the user. The physical wellness value may indicate the level of physical wellness of the user.

The processors 300 are configured to determine a mental wellness value for the user. In an example, the mental wellness value may be determined based on the set of health parameters and a set of mental physical wellness thresholds. Specifically, the mental wellness value may be determined based on the breathing data, the activity data, the posture data, the sleep pattern data and the ambience data of the user and the predefined mental wellness thresholds indicating healthy or normal values for the health parameters. The mental wellness value is associated with a stress condition of the user. The mental wellness value may indicate a level of mental wellness of the user.

The processors 300 are configured to identify a risk factor associated with a health of the user based on the physical wellness value and the mental wellness value. For example, the risk factor may indicate a risk or a health condition that the user may be facing. In an example, the risk factor may be associated with cardiac health or stress of the user. For example, if the physical wellness value and/or the mental wellness value are less than a predefined threshold, then the risk factor may be identified.

Further, the processors 300 are configured to provide a remedial procedure in real-time designed to at least mitigate the identified risk factor. For example, the remedial procedure may include an activity or a remedial process that when performed by the user, may enable the user to normalize their health parameters. For example, the remedial procedure may enable the user to normalize their breathing pattern, breathing rate, cardiac health, sleep pattern, posture, etc. leading to normalization of a HRV and/or a blood pressure of the user.

In an example, the remedial procedure may be provided to the user as a notification, for example, on a display of a user device associated with the user. For example, the remedial procedure is a time-based remedial process that is triggered based on the identification of the risk factor. To this end, as soon as the risk factor is identified for the user, during a period of time, a display of remedial process is triggered on the user device. This may enable the user to become aware of the risk factor as well as monitor and resolve such risk factor by following or performing the remedial procedure.

Pursuant to an example of the present disclosure, the remedial procedure is determined to regulate the HRV and/or the blood pressure of the user. In an example, the determined remedial procedure is associated with a resonant breathing activity. For example, the resonant breathing activity may be a slow deep breathing activity that activates calming nerves that are located towards lower lobes of lungs of the user. The resonant breathing activity may also optimize heart rate and calms mind of the user.

Further, by doing the resonant breathing activity, heart rate variability of the user may reach a maximum. As may be understood, HRV is one of the best biomarkers for gauging physical and mental health. To this end, by performing the resonant breathing activity, such as slow deep breathing at 6-8 bpm, the HRV of the user may be normalized or increased and the blood pressure of the user reduced. It may be noted that HRV of the user may be important for gauging at physical and mental health of the user closely. Further, normalizing the HRV of the user may improve health of the user.

For example, the sensor data may be continually received during a time period when the user is performing the remedial procedure or the resonant breathing activity. Subsequently, the sensor data may be used to closely look at physical and mental health of the user.

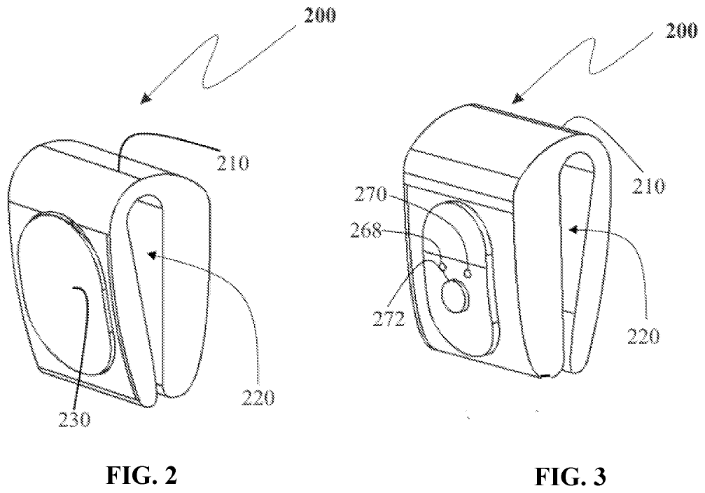

FIGS. 2, 3 and 4 illustrate the wearable device 200, in accordance with an example embodiment. The wearable device 200 comprises a casing 210 that protects the wearable device 200 to protect it from damage. Further, the wearable device 200 may be configured in a form of a clip, a strap, or any sticking material, which can be clipped or attached to a garment or undergarment of a user, specifically undergarments like, briefs, bra, panties, or any other similar undergarment. In an example, the wearable device 200 may be configured within a Velcro pattern material that may stick on garments or undergarments of the user. The wearable device 200 includes a cavity 220 for holding or hooking the wearable device 200 on the clothes of a user as shown in FIG. 7A, FIG. 7B, and FIG. 7C. Specifically, the cavity 220 is for hooking and holding the wearable device 200 on the clothes of the user in such a way that the wearable device 200 is capable of sensing certain parameters of a body of the user for collecting inputs therefrom. Further, an actuator 230 is provided on the wearable device 200 for actuating the wearable device 200 for performing sensing and other related operations. Specifically, the actuator 230 is facing towards the user's body and attached tightly to sense, for example, force or pressure applied by the user's body on the wearable device 200. A power source 240 such as a battery is placed inside the wearable device 200. The power source 240 may be a removable and rechargeable power source. The power source 240 provides power to all the elements of the wearable device 200 for their functioning.

Referring to FIG. 3, the wearable device 200 may include one or more sensors (not shown). The one or more sensors facilitate in sensing a set of health parameters, such as breathing data, activity data, posture data, sleep pattern data and ambience data, associated with the user. The set of health parameters improves the accuracy of determining physical and mental wellness or performance of the user by indicating health parameters that affect their physical and mental wellness. Specifically, the sensors may include, but are not limited to, a light sensor 268, a noise sensor 270, and a temperature sensor 272.

Referring to FIG. 1, the sensors of the wearable device 200 may also include, but is not limited to, an accelerometer 262, a gyroscope 264, and a magnetometer 266. For example, the sensors of the wearable device 200 are placed on a PCB 280.

FIG. 5 illustrates a sectional view of the wearable device 200, in accordance with an embodiment. Moreover, the one or more sensors of the wearable device 200 may also include, for example, a sensor strain gauge 260.

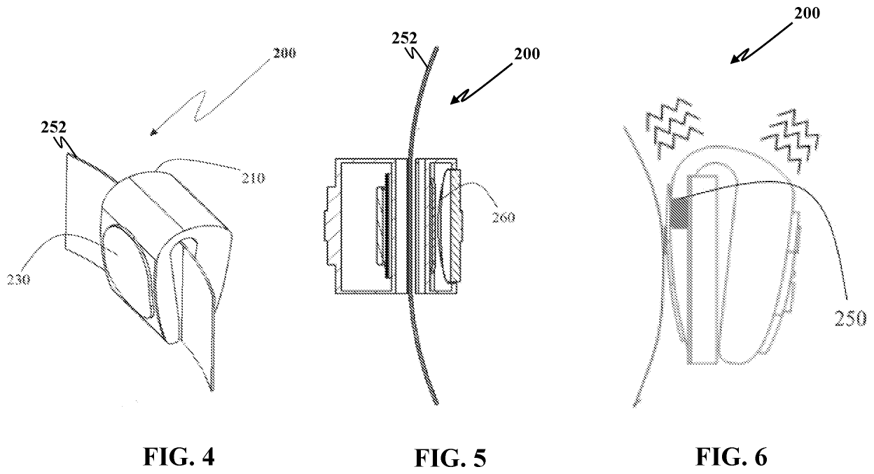

Further, FIG. 4 and FIG. 5 show a cloth 252 clamped by the wearable device 200 for attaching the wearable device to, for example, the clothes of the user.

Referring to FIG. 6, there is shown an arrangement of the wearable device 200 in a vibrating mode, in accordance with an embodiment. In particular, the wearable device 200 may include a vibrating motor 250 for sending an indicator or a response, such as a haptic response, to the user. In an example, the vibrating motor 250 may be triggered by the processors 300.

Some embodiments of the present disclosure are based on an understanding that an important factor that determines the mental wellness and the physical wellness of the user is their breathing pattern or breathing data. In an example, the breathing pattern is measured and categorized in various states such as an attentive state, a composed state, a stressed state, a depressed state and the like. It may be obvious to a person skilled in the art to measure the breathing pattern in some other obvious states. In accordance with the present embodiment, the breathing pattern or the breathing data is sensed by using the strain gauge 260 and the accelerometer 262. The strain gauge 260 and the accelerometer 262 may be used together to increase data accuracy of the breathing pattern or the sensed breathing data.

FIGS. 7A, 7B and 7C illustrate various positions for arranging the wearable device associated with a user, in accordance with various embodiments. As shown in FIG. 7A, the wearable device 200 is worn by a male user near their waist. As shown in FIG. 7B, the wearable device 200 is worn by a female user on their clothing at their waist, or on a torso near their breast on a bra. As shown in FIG. 7C, the wearable device 200 is positioned on a pillow of a user on which the user is resting their head while sleeping.

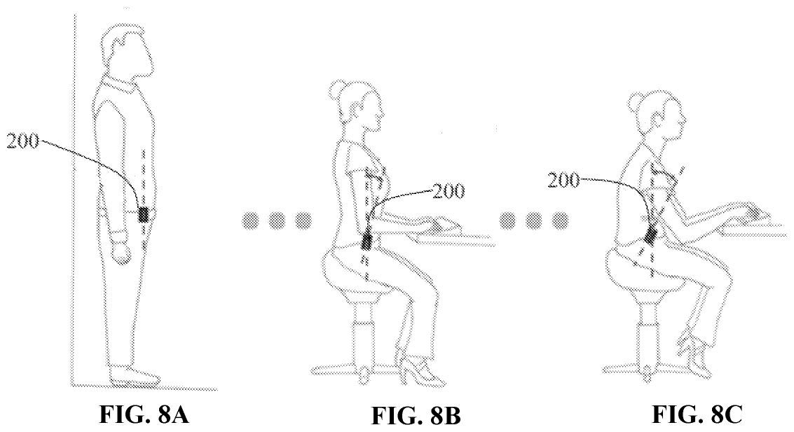

FIGS. 8A, 8B, and 8C illustrate various postures measured by the wearable device for calibration, in accordance with various embodiments. As shown in FIG. 8A, a posture of the user indicates standing straight. As shown in FIG. 8B, a posture of the user indicates sitting straight. As shown in FIG. 8C, a posture of the user indicates sitting while slouching.

Different postures of the user such as postures while sitting, standing, etc. as shown in FIGS. 8A, 8B and 8C may be sensed by the accelerometer 262, the gyroscope 264, the magnetometer 266 and the strain gauge 260.

The user has to feed the system 100 with certain user data such as name, age, gender, height, weight. Further, posture data, i.e., different postures the user is calibrated by the system 100.

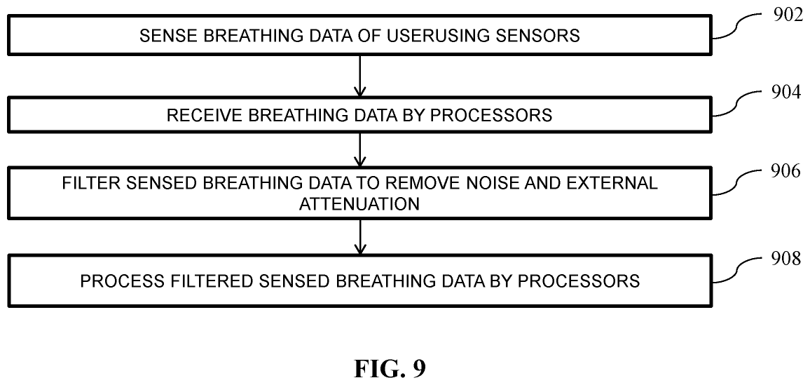

FIG. 9 illustrates a flowchart of working of the strain gauge 260 and the accelerometer 262 for sensing breathing data of the user, in accordance with an embodiment. Specifically, at 902, sensors may sense breathing data of the user. For example, the strain gauge 260 and the accelerometer 262 may sense health parameters of the user. In this regard, the wearable device 200 may be worn on a torso of the user. Thereafter, the sensors sense the health parameters. For example, the strain gauge 260 and the accelerometer 262 senses breathing data of the user by measuring, for example, torso movement while inhaling and exhaling of the user to capture breathing data. The breathing data may be sensed by measuring a change in values of the strain gauge 260 and the accelerometer 262. In another example, sensors such as the accelerometer 262, the gyroscope 264, the magnetometer 266 and the strain gauge 260 are used for sensing activity data, such as movements, rotations and activities of the user.

The strain gauge 260 may have a linear response for applied loads. Further, the strain gauge 260 is adapted to capture force and variations in the force applied to the strain gauge 260 more accurately. Thus, using the strain gauge 260 leads to higher data accuracy in sensing torso movements in a larger load for better results.

Specifically, in the present embodiment, the strain gauge 260 is used for sensing a pressure and a force applied thereon by movement of the user's body during inhaling and exhaling process or during bending. Also, the strain gauge 260 is adapted to sense the applied force, pressure on the wearable device 200 during inhale and exhale process to improve the accuracy of the breathing data. Further, the accelerometer 262 is configured to detect and monitor linear movements of the wearable device 200, thereby linear movements of the user's body.

At 904, the processors 300 may receive the sensed breathing data. The breathing data sensed by the sensors is sent to the processors 300 of the system 100.

At 906, the breathing data sensed by the sensors may be filtered to remove noise & external attenuation and converted from analog to digital by the processors 300 of the system 100.

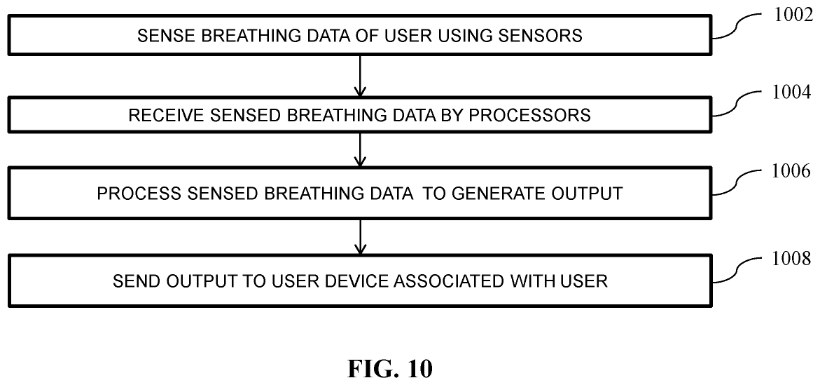

To this end, the sensors of the wearable device 200 may sense other health parameters such as state of mind data, activity data, posture data, sleep pattern data, and ambience data. Details of sensing other health parameters are described in conjunction with FIGS. 10, 11, 12 and 13.

At 908, such filtered breathing data associated with the user, may be processed by the processors 300 of the system 100, thereon. Such data may be used to determine the physical wellness value and/or the mental wellness value of the user.

Referring now to FIG. 10, a working flowchart for processing breathing data of the user is illustrated, in accordance with an embodiment.

At 1002, breathing data of the user is sensed by sensors. For example, the strain gauge 260 and the accelerometer 262 senses breathing of the user by measuring the torso movement of the user while inhaling and exhaling. The torso movement may be measured by measuring a change in values of the strain gauge 260 and the accelerometer 262. Sensor's values change from an initial value, such as a threshold, to other values, when inhaling, exhaling, and rest positions are sensed. For example, the sensors may sense health parameters of the user periodically and send a set of health parameters to the processors 300 in every, for example, minute, 2 minutes, 5 minutes, 10 minutes, 30 minutes, and so forth. In an example, the breathing data of the user may include a number of breaths per minute performed by the user.

At 1004, the sensed breathing data from the sensors is received by the processors 300 for processing.

At 1006, the processors 300 process the sensed breathing data to generate an output. In an example, the processors 300 may use the sensed breathing data to generate a physical wellness value and/or a mental wellness value for the user. It may be noted that the use of only the breathing data for determining physical wellness value and the mental wellness value is only exemplary and should not be construed as a limitation. In other embodiments, the physical wellness value and the mental wellness value of the user may be determined using different health parameters, such as breathing data, state of mind data, activity data, posture data, sleep pattern data, and ambience data.

In an example, the strain gauge 260 and the accelerometer 262 converts pressure and movement signal received from an actuator to an electrical signal. This electrical signal is filtered using combination of RC filters for noise reduction and then given to an analog to digital converter of the microprocessor. And further, the filtered data may be converted from analog to digital to determine the physical wellness value and the mental wellness value for the user. Internal signal processing is done in a microprocessor and transferred to an internal communication protocol for transferring data to an algorithm of the processors 300. Furthermore, the processed data from the processors is sent to the user device in real time. The user device may be a mobile phone or a smartphone or a computer or a tablet or any other similar devices which can synchronize the data from the wearable device 200.

In an example, the sensed breathing data may be compared with a predefined threshold to generate the physical wellness value and the mental wellness value. For example, the physical wellness value and the mental wellness value may indicate a physical health condition or physical wellness, and a mental health condition or mental wellness, respectively, of the user.

In an example, the processors 300 may periodically determine the physical wellness value and the mental wellness value of the user. Subsequently, based on the physical wellness value and the mental wellness value of the user, the system 100 or the processors 300 may monitor physical wellness and/or mental wellness of the user. Based on the monitoring of the physical wellness and/or the mental wellness of the user, the output for the user may be generated.

Continuing further, at 1008, output may be sent to a user device associated with the user. In an example, the output includes an indicator for the user in real-time. For example, the indicator relates to breathing or stress that may be faced or experienced by the user. For example, the indicator relating to the breathing or stress may be displayed on a display of the user device to inform the user regarding breathing pattern or level of stress experienced by the user during a particular time period. In an example, if breathing data of the user indicates that a number of breaths of the user may be abnormal, or the physical wellness value of the user is low, then the physical wellness and the mental wellness of the user may be poor. Subsequently, the output may include an indicator depicting decrease in the physical and wellness the mental wellness of the user.

In an example, the output is converted from hexadecimal to decimal. The decimal data is filtered again for refining one or more waveforms of breathing data of the user. In an example, the waveform may indicate breathing pattern of the user, for example, when inhaling, exhaling, at rest, stressed, etc. Waveform of the breathing pattern is further passed through a mathematical model for calibrating the breathing data and to generate breath per minute (bpm) value.

Based on periodic measurements of the sensors, the sensed health parameters may be stored. Moreover, the sensed health parameters may be correlated to a state of mind of the user. In an example, the state of mind data is compared with a mental wellness threshold to determine the mental wellness value of the user, and breathing data or bpm value is compared with a physical wellness threshold to determine the physical wellness value of the user. For example, the sensed health parameters, such as breathing data, along with other data, such as a physical wellness value during a given period of time, a mental wellness value during the given period of time, generated output, waveforms, indicator, etc. may be stored in the cloud storage 400.

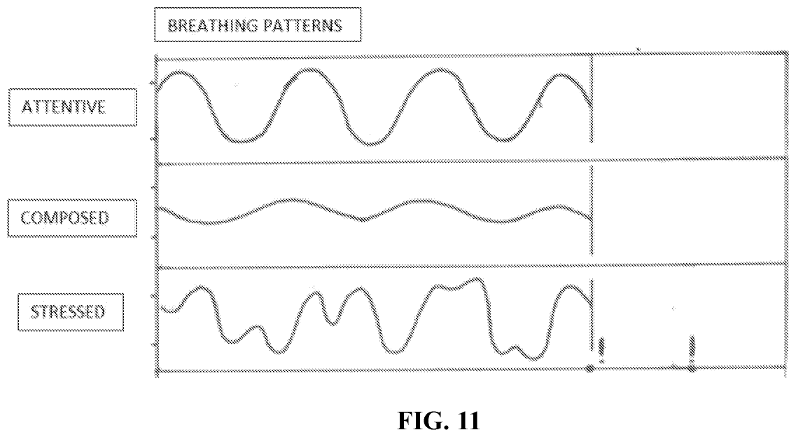

FIG. 11 illustrates a waveform of a breathing pattern of the user, in accordance with an embodiment. In an example, the mental state, such as attentive state, composed or relaxed state, and stressed state, of the user is defined by the waveform of the breathing pattern. Based on the breathing data or the breathing pattern, the mental wellness value of the user may be determined.

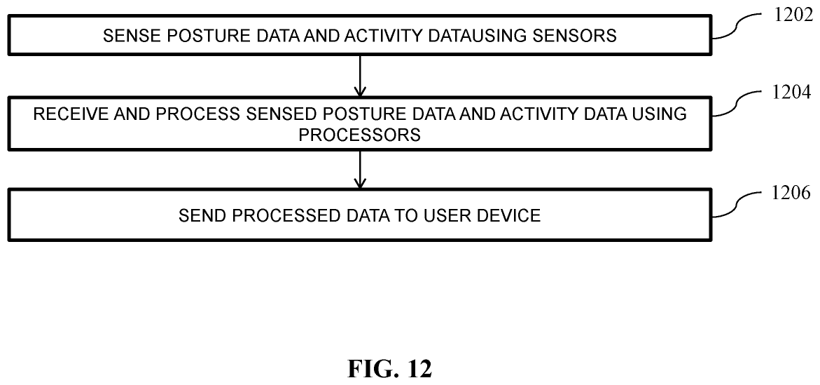

FIG. 12 shows a working flowchart of processing posture data and activity data of the user, in accordance with an embodiment. In accordance with an example, the posture data of the user is calibrated manually and fed to the system 100 as a reference data. For every user, the posture data may be calibrated in a straight standing posture state and a straight sitting posture state. It may be obvious to a person skilled in the art to calibrate posture data in other states.

In accordance with an example, a predefined posture of the user is calibrated manually and fed to the system 100 as a reference data. For every user, the predefined posture may be calibrated in a straight standing posture state and a straight sitting posture state. It may be obvious to a person skilled in the art to calibrate posture data in other states. For example, the user may feed the system 100 with data such as name, age, gender, height, weight, etc. Further, different postures, such as the predefined posture may be calibrated in various states such as slouching, standing straight and sitting straight. It may be obvious to a person skilled in the art to provide other calibrated data in various postures of the user which may bring an effect to user's lifestyle for precise calculation of a stress factor or stress level of the user.

Specifically, the posture data is sensed by the accelerometer 262, the gyroscope 264, the magnetometer 266 and the strain gauge 260. The calibrated predefined posture may be set by the user before they start using the wearable device 200.

In an example, at 1202, posture data and activity data associated with the user is sensed by sensors. In this regard, changes in values of sensors are detected. For example, change in values of the accelerometer 262, the gyroscope 264, the magnetometer 266 and the strain gauge 260 after movement, rotation, and activities of the user may be sensed by the corresponding sensors. For example, such changes may indicate a change in a posture of the user or an activity performed by the user.

Furthermore, in the present embodiment, the activity data and the posture data of the user may also be monitored for determining a usage of energy or calorie by the user. The activity data indicating activities, such as walking, standing, running and the like are monitored using the accelerometer 262, the magnetometer 266 and the gyroscope 264.

At 1204, the sensed posture data and the activity data are received by the processors 300 from sensors, such as the accelerometer 262, the gyroscope 264, the magnetometer 266 and the strain gauge 260. The sensed posture data and the activity data is received in the form of a digital signal and processed. In an example, the digital signal indicating the activity data and the posture data of the user is processed to generate physical wellness value and/or mental wellness value of the user. The physical wellness value and/or mental wellness value of the user may also be determined using other sensor data, such as breathing data, ambience data, sleep pattern data, state of mind data, etc.

In an example, during processing, the physical wellness value and/or mental wellness value of the user is compared with the physical wellness threshold and/or the mental wellness threshold, respectively. In an example, the physical wellness threshold and/or the mental wellness threshold may be determined based on the calibrated predefined posture and/or historical data relating to posture of the user. For example, a posture of the user is compared with predefined thresholds relating to sitting, standing, slouching, walking and the like, of the user. In an example, if the posture data indicates that a posture of the user is slouching then the determined physical wellness value may be low.

At 1206, processed data indicating posture data and activity data of the user is sent wirelessly to the user device. In an example, an output is generated for the user to notify and alert the user in real-time about the posture data, the activity data, the determined physical wellness value, the determined mental wellness value, etc. The output may also give guidance to the user regarding their activity data and posture data. For example, if the determined physical wellness value is low due to slouching, then the output may include guidance to the user regarding correction of posture.

The user device may be configured to displaying the indicator or the output received from the processors 300. In an example, a software application associated with the system 100 may be operably configured inside the user device. Specifically, the wearable device 200 is communicating wirelessly via Bluetooth or near field communication or Wi-Fi communication protocol with the processors 300 and the user device.

Further, the processors 300 may also receive the sleep pattern data and the ambience data around the user. The processors 300 may process the sleep pattern data and the ambience data. In an example, sleep pattern of the user is monitored for sensing sleep pattern data, such as a quality of sleep that the user gets on a daily basis.

It may be noted that the processors 300 may process the set of health parameters received from different sensors of the wearable device 200 along with other historical data of the user. The processed data may include, for example, calculated physical wellness value and mental wellness value for the user. Further, the processed and calculated data from the processors 300 may be stored on the cloud storage 400 periodically. Specifically, the breathing data, posture data, activity data, sleep pattern data and ambience data may be stored on the cloud storage 400.

In an example, the processors may receive the set of health parameters as sensed data from the wearable device 200. The set of health parameters may be used to calculate the mental wellness value and the physical wellness value of the user, and also risk factor of the ser. Subsequently, notifications are sent to the user in real time. In an example, the processors 300 may output a set of instructions to display on the user device. Such set of instructions may relate to the resonant breathing activity to enable the user to improve their mental and physical health.

In an example, the received data and the processed data and the generated data by the processors 300 may be stored within the cloud storage 400. To this end, the processors 300 may also be configured to process the historical data of historical mental wellness values and historical physical wellness values.

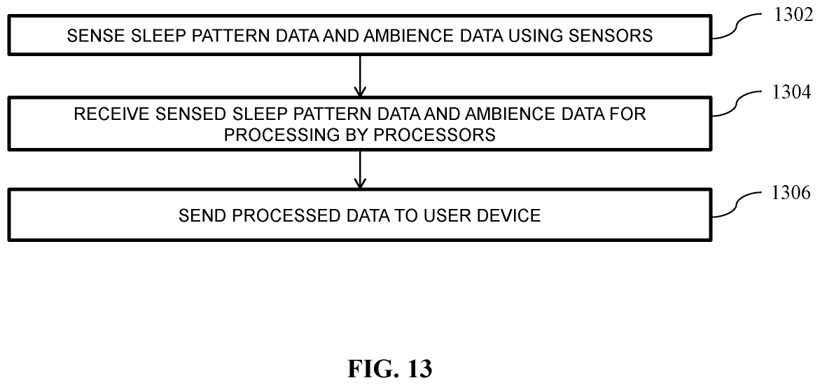

FIG. 13 shows a working flowchart for identifying sleep pattern data and ambience data around the user in the night, in accordance with an embodiment. In this regard, at 1302, sensors may sense sleep pattern data and ambience data. For example, sensors such as the accelerometer 262, the gyroscope 264, and the magnetometer 266 provided in the wearable device 200 are used to sense user's sleeping directions and movements during sleep. Moreover, sensors such as the light sensor 268, the noise sensor 270, and the temperature sensor 272 are used to sense ambience data around the user during sleep.

At 1304, the processors 300 may receive the sensed sleep pattern data and the ambience data for processing further. The received sleep pattern data and the ambience data is analyzed and compared with a corresponding threshold to determine the sleep pattern and sleep quality of the user. Such data may be used to determine the physical wellness value and/or the mental wellness value of the user. Details of processing the health parameters to determine the physical wellness value and/or the mental wellness value of the user are described in, for example, FIG. 10 and FIG. 12.

At 1306, processed data indicating sleep pattern and quality of sleep of the user is sent wirelessly to the user device. In an example, an output is generated for the user to notify and alert the user in real-time about the sleep pattern data, the ambience data, the determined physical wellness value, the determined mental wellness value, the sleep pattern, the quality of sleep etc.

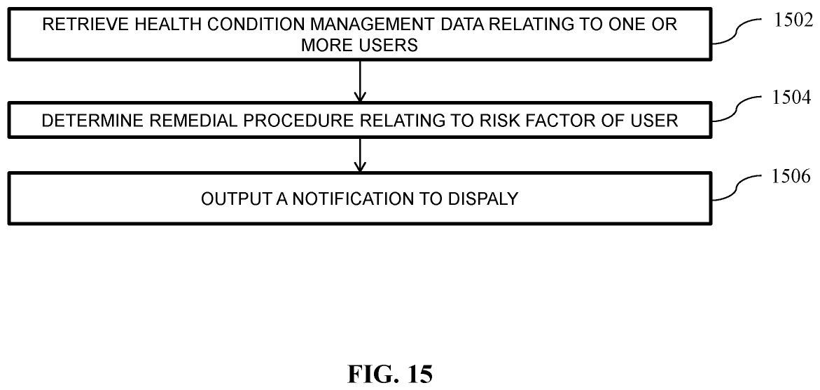

In an example, the processors 300 may provide notification and alert about the sleep quality and sleep ambience condition, completion of sleep cycle and the like through an application associated with the system 100 installed on the user device. In an example, the notification to the user may also include suggested real-time action required to be performed by the user. The notification may also provide suggested actions based on multiple data points from wider data sets of the user and other such users. Details for determining suggested actions are described in conjunction with, for example, FIG. 15.

To this end, various health parameters, such as breathing pattern, activity, ambience, sleep pattern, quality of sleep, posture, etc. may be sensed by the sensors of the wearable device 200. The sensed data, i.e., the set of health parameters may be processed to determine various features relating to the physical health and the mental health of the user. Subsequently, the sensed health parameters associated with the user may be processed to determine physical wellness value and/or the mental wellness value of the user.

Further, the wearable device 200 may include a GPS for determining a location of the user while the sensed data is being processed. The geographical location of the user may be stamped to the corresponding sensed data sensed during a particular time period. The geographic location along with the sensed data may be stored in the cloud storage 400. In an example, the geographical location information may be used for evaluating location wise physical wellness value and mental wellness value, i.e., performance and stress of the user.

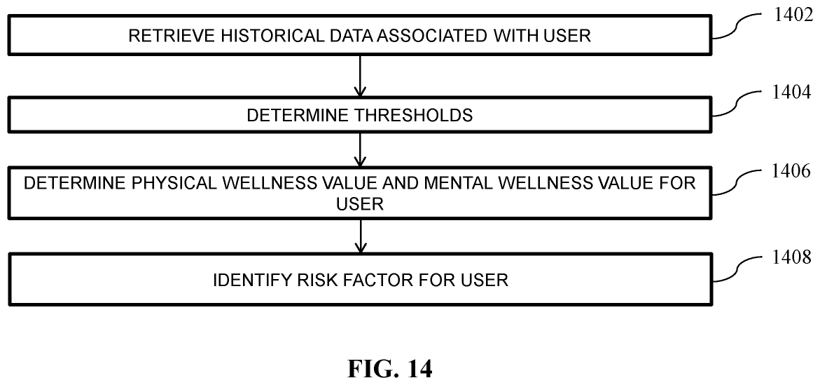

FIG. 14 illustrates a working flowchart for identifying a risk factor for the user, in accordance with an embodiment. The historical data may be processed to identify risks or health condition of the user and provide provides suggestions to improve the mental wellness and the physical wellness of the user.

In this regard, at 1402, historical data associated with the user is retrieved. In an example, the processors 300 may retrieve a set of historical physical wellness values associated with the user, and a set of historical mental wellness values associated with the u ser.

At 1404, thresholds for the user are determined. In an example, the processors 300 may determine a set of predefined physical wellness thresholds and a set of predefined mental wellness thresholds based on normal or healthy range of health parameters. The set of predefined physical wellness thresholds and the set of predefined mental wellness thresholds may indicate normal or ideal range for health parameters indicating normal or good physical and mental health.

At 1406, a physical wellness value and a mental wellness value for the user are determined. In an example, the physical wellness value for the user is determined based on a comparison between the set of health parameters, such as breathing pattern or breathing rate, and the set of predefined physical wellness thresholds, such as normal breaths per minute of user. Similarly, the mental wellness value for the user is determined based on a comparison between the set of health parameters, such as posture, breathing pattern, sleep pattern, quality of sleep, etc. and the set of predefined mental wellness thresholds, such as normal hours of sleep, REM cycles, sleep pattern, posture, exercise or workout time etc.

In an example, the set of historical physical wellness values and the set of historical mental wellness values of the user may also be used to determine the physical wellness value and the mental wellness value of the user.

At 1408, a risk factor for the user is identified. Based on the physical wellness value and the mental wellness value for the user, the risk factor for the user may be identified. For example, the risk factor may be an abnormal health condition experienced by the user. The risk factor may relate to, for example, the heart of the user, lungs of the user, stress or experience by the user, sleep of the user, etc. In an example, the risk factor may be determined based on the set of historical physical wellness values and the set of historical mental wellness values. For example, if the historical physical wellness values and/or the set of historical mental wellness values may indicate an occurrence of an abnormal health parameter, then such data may be used to predict occurrence of a risk factor and/or a future occurrence of a disease.

FIG. 15 illustrates a flowchart of generating a notification for providing a remedial procedure to a user, in accordance with an embodiment.

In this regard, at 1502, health condition management data relating to one or more users is retrieved. The one or more users may be other users that may also have the risk factor identified for the user. For example, the health condition management data may indicate one or more steps, actions or tasks performed by the other users to address the risk factor.

At 1504, the remedial procedure relating to the risk factor of the user is determined. The remedial procedure may be determined based on, for example, the health condition management data, the physical wellness value, the mental wellness value, the set of historical mental wellness values of the user, and the set of physical wellness values of the user. The remedial procedure may include personalized wellness data for the user. Personalized wellness data may include, for example, personalized diet chart, personalized shopping list, personalized workout plan, personalized doctor recommendations and so forth. The personalized wellness data along with the resonant breathing activity may assist the user in improving their health and reducing the risk factor. In an example, the processors 300 may identify patterns between the user and other users to identify a relation between the risk factor, future health complications and wellness activities or recommendations. For example, the risk factor identified for the user may be stress. In such a case, the processors 300 may process may historical data of stress relating to the one or more other users determine suggestions to reduce stress.

At 1506, a notification is output to display. For example, the processors 300 may output the notification indicating the remedial procedure for improving the physical wellness value and/or the mental wellness value of the user. The notification may be displayed on the user device associated with the user. The notification may assist the user in performing the resonant breathing activity, reduce stress, improve cardiac health, and other personalized wellness recommendations. For example, the notification may include a set of instructions indicating step-by-step process to enable the user to perform the resonant breathing activity and other personalized wellness recommendations. In an example, the notification may include suggestions to improve the mental wellness and the physical wellness and/or suggestions to improve the user's lifestyle.

In an example, the system 100 may establish a two-way communication with the user device. The communication is established for sending the output notification to the user which can be displayed on the user device. The electronic device 300 and the cloud storage 400 can communicate through internet.

In an example, the processors 300 may be configured to receive and aggregate data, such as set of health parameters, risk factor, physical wellness value, mental wellness value, notification, etc. from various users wearing their wearable devices, such as the wearable device 200. The aggregated data may be displayed on a user device or any such terminals.

In one embodiment, user groups may be created on the cloud storage 400, and the aggregate data may be displayed on the user device (such as mobile, computer, tablet and the like) through the system 100. This group may be of a company, a family or any other group.

In accordance with an example embodiment, the processors 300 may be implemented using an artificial intelligence based neural network. For example, the neural network of the processors 300 may be configured to compare and analyze the set of health parameters. In an example, the neural network may be configured to determine the physical wellness value and the mental wellness value to determine the risk factor for the user. For example, the neural network may be configured to determine mental state, or the mental wellness value of the user based on stress factor of the user. The stress factor may be determined based on, for example, the breathing data, the posture data, the activity data, the sleep pattern data, etc. Similarly, based on the set of health parameters, the neural network may determine physical state or the physical wellness value of the user. Further, the neural network of the processors 300 may also be configured to determine if the identified risk factor of the user is associated with a future health condition.

In an example, fine-tuning of the data for the neural network is done on the cloud storage 400. Long-term reports of the users are generated by the processors 300, for example, using the neural network.

For example, the user may switch the wearable device 200 to a night mode before sleeping. During the night mode, the ambience data around the user such as light, noise, and temperature is sensed by using, for example, the light sensor 268, the noise sensor 270, and the temperature sensor 272 respectively. Moreover, sleep pattern data is also sensed. Further, the ambience data and the sleep pattern data is sent to the processors 300, which further processes and analyses the ambience data and the sleep pattern data along with other sensor data indicating the set of health parameters.

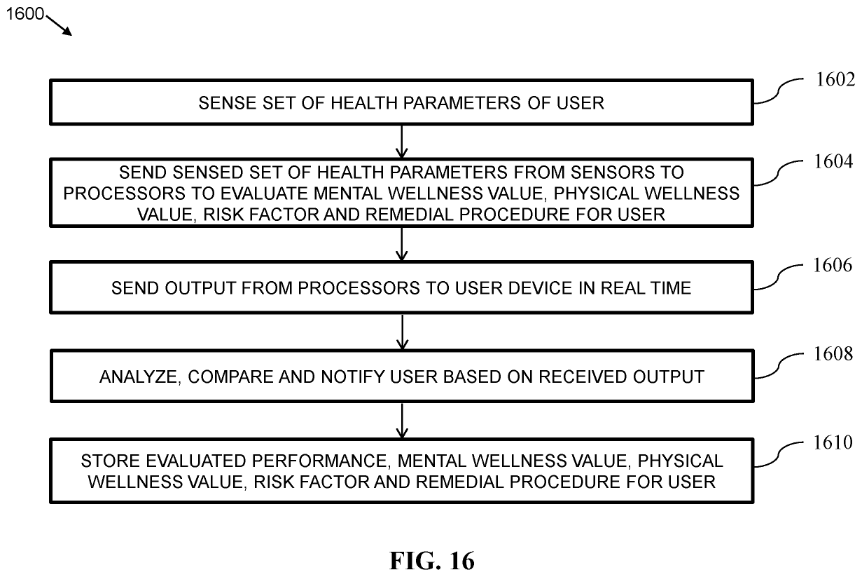

FIG. 16 shows a method 1600 for monitoring human performance, in accordance with an embodiment. For the sake of brevity, the method 1600 is explained in conjunction with the system 100 and FIGS. 1-15.

At 1602, the set of health parameters of the user is sensed by sensors of the wearable device 200. The set of health parameters may include, for example, breathing data, activity data, posture data, sleep pattern data, and ambience data. In one embodiment, geographical location of the user corresponding to the sensed set of health parameters is stored and used for evaluating location wise physical wellness and mental wellness of the user.

At 1604, sensed set of health parameters is sent from the sensors of the wearable device 200 to the processors 300 of the system 100 in real time. The processors 300 are configured to process the set of health parameters to evaluate performance, the mental wellness and the physical wellness of the user. The processors 300 may be configured to identify one or more risk factors for the user. For example, based on the set of health parameters, the processors may identify if the user is experiencing stress, cardiac issues, sleeping issues, breathing issues, anxiety, etc. Moreover, the processors 300 are configured to determine and output a remedial procedure to assist the user to resolve or fully or partially mitigate the risk factor.

At 1606, the output from the processors 300 is sent to the user device in real time.

Further, at 1608, the received output is analyzed and compared with a calibrated and fed data such as name, gender, age, weight and the like of the user. The received output is then notified to the user along with in real time through a display of the user device.

In an example, the output from the processors 300 may also be sent to a caretaker, a health professional, and/or a person associated with an organization of the user. In this manner, organizations may be able to monitor physical as well as mental wellness of their employees. Further, health professional of the user may be able to analyze complete data of the user to provide medical advisory, such as medicines, lifestyle supplements, etc.

In an example, along with the set of health parameters, the processors 300 may retrieve and process historical data of the user and historical management data of other users having risk factor to identify patterns and accordingly identify and provide the remedial procedure to improve the user's lifestyle, the mental wellness and the physical wellness.

At 1610, the evaluated performance, the mental wellness value, the physical wellness value, the risk factor and the remedial procedure for the user may be stored in the cloud storage 400. Such stored data may be used as historical data of the user to identify risk factor for the user at a later stage, for example, in future.

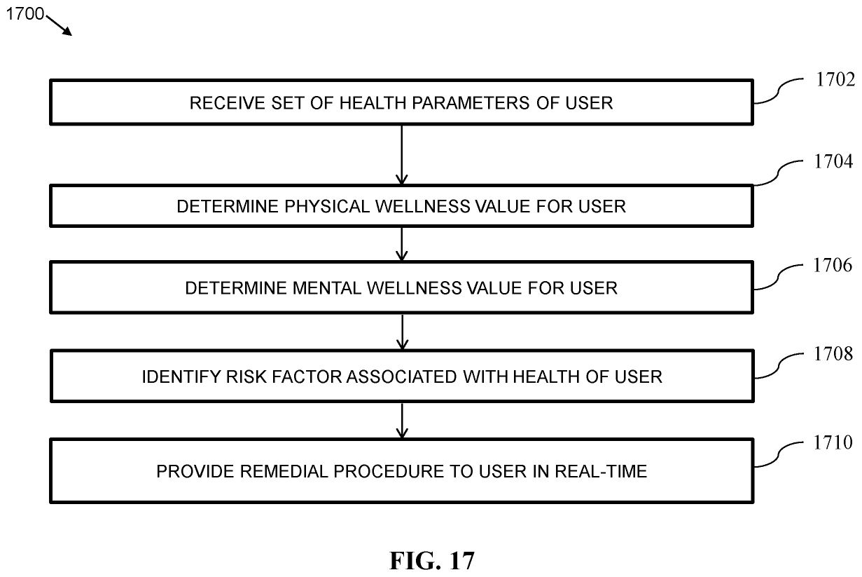

FIG. 17 shows a method 1700 for providing a remedial procedure to a user, in accordance with an embodiment. For the sake of brevity, the method 1700 is explained in conjunction with the system 100 and FIGS. 1-16.

At 1702, the set of health parameters of the user is received from the one or more sensors. In an example, the sensors may be positioned within the wearable device 200. For example, the wearable device 200 may be worn or positioned at a torso of the user, or a waist of the user. For example, the sensors of the wearable device may be positioned against body of the user to measure, monitor and track health parameters, such as blood pressure, number of steps, activities steps, temperature, breathing rate, heart rate, heart rate variability, sleep pattern, etc. of the user. The sensors of the wearable device may include, for example, the light sensor 268, the noise sensor 270, the temperature sensor 272, the accelerometer 262, the gyroscope 264, the magnetometer 266, or the strain gauge 260. For example, the set of health parameters sensed by the sensors may include, but is not limited to, breathing data, state of mind data, activity data, posture data, sleeping pattern data, and ambience data.

At 1704, a physical wellness value for the user is determined. The physical wellness value may be determined based on the received set of health parameters and a set of predefined physical wellness thresholds.

In an example, the set of predefined physical wellness thresholds may be determined for the user based on predefined normal or ideal health parameters. Further, based on a comparison between values of the set of health parameters and values of the corresponding predefined physical wellness thresholds, the physical wellness value for the user may be determined. In an example, the physical wellness value may be associated with a breathing pattern, or a heart rate, of the user. For example, the physical wellness value may be indicative of a level or state of physical health of the user.

In an example, a set of historical physical wellness values associated with the user may be retrieved from, for example, the cloud storage 400. Further, the physical wellness value for the user may be determined based on the set of health parameters and the set of historical physical wellness values. The set of historical physical wellness values may indicate previously calculated physical wellness values for the user.

At 1706, a mental wellness value for the user is determined. The mental wellness value may be determined based on the received set of health parameters and a set of predefined mental wellness thresholds. For example, the mental wellness value for the user may be determined based on the breathing data and the state of mind data.

In an example, the set of predefined mental wellness thresholds may be determined for the user based on predefined normal or ideal health parameters. Further, based on a comparison between values of the set of health parameters and values of the corresponding predefined mental wellness thresholds, the mental wellness value for the user may be determined. In an example, the mental wellness value may be associated with a stress condition of the user. For example, the mental wellness value may be indicative of a level or state of mental health of the user.

In an example, a set of historical mental wellness values associated with the user may be retrieved from, for example, the cloud storage 400. Further, the mental wellness value for the user may be determined based on the set of health parameters and the set of historical mental wellness values. The set of historical mental wellness values may indicate previously calculated mental wellness values for the user.

For example, the system 100 may monitor the physical wellness and/or the mental wellness of the user by determining the physical wellness value and the mental wellness value for the user periodically.

Further, at 1708, the risk factor associated with a health of the user is identified based on the physical wellness value and the mental wellness value. The risk factor may also be identified based on the set of historical mental wellness values and the set of historical physical wellness values of the user. The risk factor may indicate a chance or a likelihood of occurrence of a health condition that may harm or otherwise affect the health of the user. For example, the risk factor may be related to heart of the user, heart rate, lung capacity, free radicals, stress regulating system of the user, etc.

In an example, the processors 300 may be configured to retrieve health condition management data relating to one or more users. The one or more users may be other users having the risk factor identified for the user. For example, the processors 300 may identify patterns between health parameters of the user and health parameters of the other users. Based on the identified patterns, the processors 300 may determine the risk factor for the user.

At 1710, a remedial procedure is provided to the user in real-time based on the identified risk factor. The remedial procedure is designed to at least mitigate the identified risk factor. In an example, the processors 300 may be configured to determine the remedial procedure relating to the risk factor based on the health condition management data associated with the other users having the risk factor, the physical wellness value of the user, the mental wellness value of the user, the identified risk factor, the set of historical mental wellness values of the user, and the set of physical wellness values of the user. For example, the remedial procedure is determined based on heart rate variability (HRV) of the user. In an example, the determined remedial procedure is associated with a resonant breathing activity. For example, the remedial procedure may be an instant relief program or activity to improve current health of the user.

The remedial procedure may be a time-based remedial process, for example, a time bound activity that is triggered based on the identification of the risk factor. For example, for different risk factors, different remedial procedures may be determined, that may have to be performed for different durations of time. In accordance with an example, the remedial procedure may be the resonant breathing activity in which the user may be asked to perform remedial process of breathing at 6-8 breaths per minute (bpm). In an example, the processors 300 may output a notification to display based on the determined remedial procedure. For example, the notification may be sent to the user device to notify and alert the user about the risk factor. The notification may also indicate the remedial procedure for improving the physical wellness value and/or the mental wellness value of the user. In certain cases, the processors 300 may also output a set of instructions to display on the user device. The set of instructions may indicate steps or ways to perform the remedial procedure, such as the resonant breathing activity. Moreover, by providing the remedial procedure as the set of instructions, the processors 300 may cause the user to perform the resonant breathing activity in order to address the risk factor.

It may be noted that the processors 300 may cause the user to perform the resonant breathing activity as heart beats optimally with consistency and high oscillations at resonant breathing of 6-8 bpm leading to high heart rate variability. Resonant breathing may also increase lung capacity, help in reduction of blood pressure, help in better control of autonomic nervous system (ANS) on cardiovascular function, bring calmness and clarity, reduce stress, and improve sleep. Further, after or during the resonant breathing activity, the HRV of the user may be used to gauge physical wellness and/or mental wellness of the user. Based on such data, the processors 300 may be able to accurately determine a disease or state of physical and mental health of the user.

In an example, the remedial procedure may also include personalized wellness data, such as diet plan, exercise plan, etc. for the user to mitigate the risk factor and/or improve lifestyle of the user.

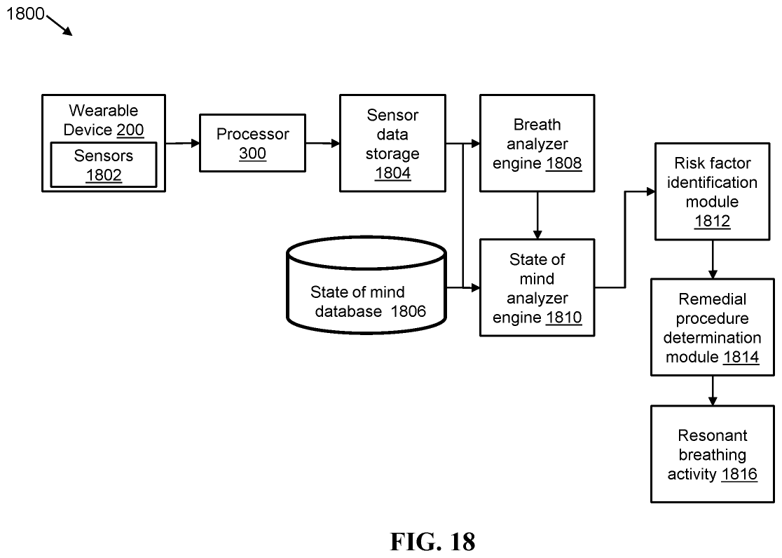

FIG. 18 illustrates a schematic diagram 1800 for performing operations of a system for providing a remedial procedure, in accordance with an example embodiment. FIG. 18 is explained in conjunction with elements of FIGS. 1-17. The schematic diagram 1800 may be implemented by the system 100. The system 100 may include the wearable device 200. The wearable device 200 may include sensors 1802, for example, the accelerometer 262, the gyroscope 264, the magnetometer 266 the light sensor 268, the noise sensor 270, and the temperature sensor 272. Details of the wearable device 200 are provided in conjunction with, for example, FIG. 1, FIG. 2, FIG. 3, FIG. 4, FIG. 5 and FIG. 6.

In particular, the sensors 1802 of the wearable device 200 may measure certain parameters of the body of the user for collecting inputs therefrom. Data collected by the sensors 1802 may be stored within the processors 300. In an example, the processors 300 may be configured to pre-process the received raw data to determine the set of health parameters of the user. The set of health parameters may include, for example, breathing data (or a breathing pattern), activity data, posture data, sleep pattern data and ambience data. Further, the processors 300 may store the collected data, i.e., the set of health parameters in a sensor data storage 1804. For example, the sensor data storage 1804 may include, for example, one or more volatile and/or non-volatile memories.

In an example, the state of mind database 1806 may store state of mind data of the user. The state of mind data may include, for example, different states of mind, such as stressed, relaxed, anxious, etc. of the user during different time periods. For example, such history of state of mind of the user may be determined based on sensed breathing data during corresponding different time periods.

Further, the historical state of mind data from the state of mind database 1806 and currently sensed data from the sensors 1802 may be provided to a breath analyzer engine 1808. For example, the breath analyzer engine 1808 may analyze the breathing data or the breathing pattern of the user to categorize the user into one of the various states such as the attentive state, the composed state, the stressed state, the depressed state, and the like. Further, the breath analyzer engine 1808 may provide the determined current state of mind of the user to a state of mind analyzer engine 1810. The state of mind analyzer engine 1810 is configured to analyze the historical state of mind data and the current state of mind of the user based on the breathing data, as well as other data, such as the activity data, the posture data, the sleep pattern data and the ambience data. The state of mind analyzer engine 1810 is configured to determine the mental wellness value of the user.

Once the state of mind of the user is determined, the physical wellness value may also be determined for the user. For example, the breath analyzer engine 1808 and the state of mind analyzer engine 1810 may provide physical wellness value and mental wellness value of the user. Further, a risk factor identification module 1812 may be configured to identify any deviation or anomaly associated with the health parameters, the physical wellness value and/or the mental wellness value of the user to identify any physical or mental problem, i.e., a risk factor for the user. In an example, the risk factor identification module 1812 may be configured to anticipate problems, health conditions, deviations, health patterns, etc. based on the state of mind data, the mental wellness value and the physical wellness value of the user.

Based on the identified risk factor, the remedial procedure determination module 1814 may be configured to identify a remedial procedure for improving a health of the user based on the risk factor. The remedial procedure may be a time-based remedial process that is triggered based on the identification of the risk factor. In an example, the remedial procedure may include a resonant breathing activity 1816. The remedial procedure of the resonant breathing activity 1816 may cause increased heart rate variability, normalizes sympathovagal balance and reduces free radicals in the body of the user. For example, the resonant breathing activity 1816 may include instructions for slow deep breathing exercise at 6-8 bpm to achieve high heart rate variability. For example, the processors 300 may be configured to monitor health, physical wellness and mental wellness of the user by periodically determining the physical wellness value and the mental wellness value of the user. Based on periodic monitoring, the processors 300 may accurately identify risk factors for the user. For example, the processors 300 may provide forewarning for heart failure related conditions. Subsequently, the remedial procedure may be determined and provided to the user.

In certain cases, the processors 300 may also output an indicator for the user in real-time. The indicator may relate to breathing, or stress, based on the physical wellness or mental wellness of the user. In an example, the processors 300 may output an indicator relating to real-time breathing rate and stress level of the user. In another example, the processors 300 may output an indicator relating to real-time state of mind, such as calm, focused, at rest, etc. of the user, for example, based on breathing pattern of the user. For example, the indicator may be a textual or visual depiction of the breath rate, the stress level and/or the state of mind of the user.

Therefore, the system 100 described in the present disclosure has an advantage of providing the wearable device 200 for monitoring human performance. The system 100 keeps real-time track of a mental and a physical health of a user. Specifically, the system 100 monitors breathing data (such as breathing rate, breathing pattern, etc.) in real-time, interprets state of mind of the user in real-time, identifies risk factor for the user, provides remedial procedure for instant relief, as well as provides forewarning for adverse health conditions. The system 100 also provides alerts or notifications to the user in real-time based on identified risk factor and remedial procedure to improve physical or mental health of the user instantly. For example, the system 100 also identifies a problem behind stress for the user in a particular period of time. Further, the system 100 can send an alert to the user when their physical wellness value or mental wellness value is low, i.e., not good. The system 100 helps the user to check breathing pattern, activity, posture, sleeping pattern and ambience. The processors 300 may process and provide data, such as remedial procedure, risk factor, indicator, notification, etc. to the user as well as to a corporate company for enhancement of work efficiency of the user as well as a group associated with the user. The work efficiency of the user may be collected through the wearable device 200 and further processed for assessment by human resource team of an organization to initiate required corrective actions. Further, the system 100 is capable to assist and monitor the user in real time. In certain cases, the system 100 may also analyze the user's personality as well as their compatibility with others, for example, based on their stress level, physical wellness, quality of sleep, etc.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the present disclosure. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the present disclosure. In this regard, for example, different combinations of elements and/or functions than those explicitly described above are also contemplated as may be set forth in some of the embodiments of the present disclosure. Although specific terms are employed herein, they are used in a generic and descriptive sense only and not for purposes of limitation.

What is claimed is:

1. A system comprising:
a wearable device configured to operate with one or more sensors to measure a set of health parameters of a user, the health parameters comprising at least one of: breathing data, state of mind data, activity data, posture data, sleep pattern data, and ambience data; and
one or more processors operably connected to the wearable device, the one or more processors being configured to:
receive the set of health parameters of the user from the one or more sensors;
determine a physical wellness value for the user based on the set of health parameters and a set of predefined physical wellness thresholds, the physical wellness value being associated with at least one of: a breathing pattern, or a heart rate, of the user;
determine a mental wellness value for the user based on the set of health parameters and a set of predefined mental wellness thresholds, the mental wellness value being associated with at least a stress condition of the user;
based on the physical wellness value and the mental wellness value, identify a risk factor associated with a health of the user; and
provide a remedial procedure in real-time designed to at least mitigate the identified risk factor.

2. The system of claim 1, wherein the remedial procedure is a time-based remedial process that is triggered based on the identification of the risk factor.

3. The system of claim 1, wherein the remedial procedure is determined to regulate at least one of: a heart rate variability (HRV), or a blood pressure, of the user, wherein the determined remedial procedure is associated with a resonant breathing activity.

4. The system of claim 3, wherein the one or more processors are further configured to:
cause the user to perform the resonant breathing activity in order to address the risk factor relating to at least one of: heart rate, heart rate variability, free radicals, or stress regulating system.

5. The system of claim 3, wherein the one or more processors are further configured to:
output a set of instructions to display, the set of instructions relating to the resonant breathing activity.

6. The system of claim 1, wherein the one or more processors are further configured to:
retrieve health condition management data relating to one or more users, the one or more users having the risk factor identified for the user;
determine the remedial procedure relating to the risk factor based on the health condition management data, the physical wellness value, the mental wellness value, a set of historical mental wellness values of the user, and a set of physical wellness values of the user, wherein the remedial procedure comprises personalized wellness data for the user; and
based on the determined remedial procedure, output a notification to display, the notification indicating the remedial procedure for improving at least one of: the physical wellness value, or the mental wellness value, of the user.

7. The system of claim 6, wherein the one or more processors are further configured to:
retrieve the set of historical physical wellness values associated with the user; and
determine the physical wellness value for the user based on the set of health parameters and the set of historical physical wellness values.

8. The system of claim 6, wherein the one or more processors are further configured to:
retrieve the set of historical mental wellness values associated with the user; and
determine the mental wellness value for the user based on the set of health parameters and the set of historical mental wellness values.

9. The system of claim 1, wherein the one or more processors are further configured to:
determine the mental wellness value for the user based on the breathing data and the state of mind data.

10. The system of claim 1, wherein the one or more processors are further configured to:
periodically determine the physical wellness value and the mental wellness value of the user;
monitor at least one of: physical wellness, or mental wellness, of the user based on at least one of: the physical wellness value, or the mental wellness value; and
output an indicator for the user in real-time, the indicator relating to at least one of: breathing, or stress, based on at least one of the: physical wellness, or mental wellness.

11. The system as claimed in claim 1, wherein the one or more sensors comprises at least one of: a light sensor, a noise sensor, a temperature sensor, an accelerometer, a gyroscope, a magnetometer, or a strain gauge.

12. The system as claimed in claim 1, wherein the wearable device is worn on a torso of the user.

13. A method comprising:
receiving a set of health parameters of the user from the one or more sensors, the health parameters comprising at least one of: breathing data, state of mind data, activity data, posture data, sleep pattern data, and ambience data;
determining a physical wellness value for the user based on the set of health parameters and a set of predefined physical wellness thresholds, the physical wellness value being associated with at least one of: a breathing pattern, or a heart rate, of the user;
determining a mental wellness value for the user based on the set of health parameters and a set of predefined mental wellness thresholds, the mental wellness value being associated with at least a stress condition of the user;
identifying a risk factor associated with a health of the user based on the physical wellness value and the mental wellness value; and
providing a remedial procedure in real-time designed to at least mitigate the identified risk factor.

14. The method of claim 13, wherein the remedial procedure is a time-based remedial process that is triggered based on the identification of the risk factor.

15. The method of claim 13, wherein the remedial procedure is determined based on heart rate variability (HRV) of the user, the determined remedial procedure being associated with a resonant breathing activity.

16. The method of claim 15, further comprising:
causing the user to perform the resonant breathing activity in order to address the risk factor relating to at least one of: heart rate, heart rate variability, free radicals, or stress regulating system.

17. The method of claim 15, further comprising:
outputting a set of instructions to display, the set of instructions relating to the resonant breathing activity.

18. The method of claim 13, further comprising:
retrieving health condition management data relating to one or more users, the one or more users having the risk factor identified for the user;
determining the remedial procedure relating to the risk factor of the user based on the health condition management data, the physical wellness value, the mental wellness value, a set of historical mental wellness values of the user, and a set of physical wellness values of the user, wherein the remedial procedure comprises personalized wellness data for the user; and
based on the determined remedial procedure, outputting a notification to display, the notification indicating the remedial procedure for improving at least one of: the physical wellness value, or the mental wellness value, of the user.

19. The method of claim 13, further comprising:
determining the mental wellness value for the user based on the breathing data and the state of mind data.

20. A computer programmable product comprising a non-transitory computer readable medium having stored thereon computer executable instructions, which when executed by one or more processors, cause the one or more processors to carry out operations comprising:
receiving a set of health parameters of the user from the one or more sensors, the health parameters comprising at least one of: breathing data, state of mind data, activity data, posture data, sleep pattern data, and ambience data;
determining a physical wellness value for the user based on the set of health parameters and a set of predefined physical wellness thresholds, the physical wellness value being associated with at least one of: a breathing pattern, or a heart rate, of the user;
determining a mental wellness value for the user based on the set of health parameters and a set of predefined mental wellness thresholds, the mental wellness value being associated with at least a stress condition of the user;
identifying a risk factor associated with a health of the user based on the physical wellness value and the mental wellness value; and
providing a remedial procedure in real-time designed to at least mitigate the identified risk factor.
